(19) United States
(12) Patent Application Publication
(10) Pub. No.: US 2004/0203096 A1
(43) Pub. Date: Oct. 14, 2004

(54) Synthesis and secretion of native recombinant lysosomal enzymes by liver

(76) Inventors: Nina Raben, Rockville, MD (US); Nina Lu, Rockville, MD (US); Bo Yan, Rockville, MD (US); Paul Plotz, Washington, DC (US); Yesenia Rivera, Bethesda, MD (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/474,163

(22) PCT Filed: Apr. 9, 2002
(86) PCT No.: PCT/US02/11119

Related U.S. Application Data
(60) Provisional application No. 60/282,422.

(51) Int. Cl. C12N 9/36; C07H 21/04

(52) U.S. Cl. 435/69.1; 435/320.1; 435/325; 435/206; 536/23.2

(57) ABSTRACT

The invention provides recombinant native lysosomal enzymes produced by liver cells, preferably in vitro, and methods of using the native recombinant lysosomal enzymes to treat enzyme deficiencies in vivo. Lysosomal enzymes, including acid alpha-glucosidase (GAA), produced by liver cells apparently undergo the post-translational modifications necessary to achieve good enzymatic activity. The resulting enzymes can be taken up by various other cells and can correct phenotypic abnormalities of distant organs with enzyme deficiencies. In certain preferred embodiments, the enzyme is GAA and the methods are especially adapted for treatment of type II glycogen storage disease in mammals, including humans.

### FIELD OF THE INVENTION

The invention generally relates to synthesis and secretion of native recombinant lysosomal enzymes, including acid alpha-glucosidase, by liver cells, and treatment of certain disorders with the native recombinant lysosomal enzymes.

### BACKGROUND

Glycogen storage disease type II (GSDII) is an autosomal recessive disorder caused by the deficiency of acid alpha-glucosidase (GM), a glycogen-degrading lysosomal enzyme. The enzyme catalyzes the hydrolysis of glycogen to glucose by cleaving both α-1,4 and α-1,6 glycosidic linkages. Complete deficiency of the enzyme results in a severe and generalized disease of infancy associated with the massive accumulation of lysosomal glycogen in almost all tissues of the body (Pompe syndrome). This most severe infantile-onset form of the disease manifests in the first months of life as hypotonia and muscle weakness, macroglossia, moderate hepatomegaly, and a rapidly progressive hypertrophic cardiomyopathy leading to cardiac failure before 2 years of age. Partial deficiency of the enzyme results in milder late-onset variants, which manifest as slowly progressive limb-girdle-like myopathy, eventually leading to death from respiratory insufficiency due to diaphragmatic involvement (reviewed in Hirschhorn, 1995).

Acid alpha-glucosidase is synthesized in the endoplasmic reticulum (ER) as a 110-kDa catalytically active precursor that undergoes extensive post-translational modifications. The glycosylated precursor is modified in the Golgi compartment by phosphorylation of mannose residues. The majority of the modified precursor containing the mannose 6-phosphate (M-6-P) groups are targeted to the lysosomal compartment where the protein is proteolytically cleaved to give rise to the final lysosomal 76 and 70 kDa forms. A portion of the newly synthesized precursor is, however, secreted, and this extracellular enzyme can be internalized by neighboring or distant cells via M-6-P receptors present on plasma membrane and on the surface of the cells (Hoefsloot et al., 1990; Wisselaar et al., 1993; Reuser et al., 1984). Partially secreted lysosomal enzymes can also be taken up by a M-6-P-independent pathway (Tsuji et al., 1998). The discovery of this secretion-recapture mechanism has provided a rationale for both cell-mediated and direct enzyme replacement therapy for GSDII as well as for other lysosomal disorders (Van der Ploeg et al., 1998, 1991; Neufeld, 1991).

Recombinant human GM has been produced in CHO cells and in transgenic rabbit milk (Fuller et al., 1995; Van Hove et al., 1996; Bijvoet et al., 1999). However, this recombinant human GM does not appear to have all of the post-translational modifications found in GM in vivo. Perhaps because of one or more of the missing post-translational modifications, recombinant human GM produced thus far does not appear to have the full activity of native human GM. Therefore, there remains a need for a method of producing recombinant GM and other lysosomal enzymes where post-translational modifications are retained; there also remains a need that such recombinant enzymes have good activity and can be used for replacement therapy.

Another approach to treating diseases resulting from insufficient GM activity is somatic cell gene therapy. A number of studies have demonstrated that skeletal muscle gene therapy can result in sustained and systemic delivery of therapeutic proteins. Skeletal muscle is an attractive target for somatic gene therapy due to its large mass (more that 40% of the human body), high vascularity, accessibility for intramuscular injections, and large capacity for protein synthesis which allows de novo synthesized proteins ready access to systemic circulation. Intramuscular injections of recombinant MV encoding murine erythropoetin or factor IX into mice resulted in persistent and stable high level secretion of the proteins (Kessler et al., 1996; Vincent-Lacaze et al., 1999; Herzog et al., 1997). Genetically engineered muscle cells expressing high level of insulin mRNA and protein were able to produce insulin in vivo after transplantation into skeletal muscle of diabetic mice, resulting in a reduction of hyperglycemia (Gros et al.,1999). Adenovirus-mediated muscle gene transfer of neurotrophin-3 produced substantial therapeutic effects in mice with progressive motor neuropathy due to high-level production of neurotrophin in injected muscle and its liberation into the systemic circulation with the factor acting peripherally (Hasse et al., 1997).

One challenge for gene therapy of diseases involving GM deficiency, such as GSDII, is that a systemic delivery of GM is absolutely critical. This conclusion is based on the fact that some affected muscles, including diaphragm, intercostal muscles, and heart in the infantile form of the disease, are not easily accessible. However, since only a small percentage of normal GM activity is required for therapeutic effect, low levels of circulating enzyme should be sufficient to provide phenotypic correction in distant organs (Reuser et al., 1995).

In vitro and ex-vivo experiments with muscle-mediated GM delivery provided encouraging results. A significant increase in the GM enzyme level (20-fold) in deficient muscle cells and the phenotypic correction of cultured skeletal muscle cells from a GSDII patient was obtained with a recombinant adenovirus containing the human gene (Nicolino et al., 1998). A high level of GM expression (30-fold increase compared to the levels in normal individuals) was also observed in GM deficient myoblasts transduced with a GAA-encoding retrovirus (Zaretsky et al., 1997). Furthermore, gene-corrected myoblasts were capable of secreting the enzyme which provided enzymatic activity to the non-transduced deficient muscle cells by both secretion and fusion mechanisms. The implication of these studies was that ex-vivo transduced myoblasts from patients could then be implanted into the muscles of affected individuals, and would then secrete the enzyme for uptake by other cells.

Despite the encouraging results of ex vivo and in vitro muscle cell studies, results of in vivo muscle-mediated GM delivery have been disappointing. Intracardiac or intramuscular administration of a recombinant adenoviral vector expressing GM (Ad-GM) into newborn rats resulted in high levels of GM expression locally; the heart, liver, contralateral muscle, and sera of these rats, however, showed no increase in GM activity (Pauly et al., 1998). Similarly, a recombinant Ad-GAA injected into muscle of GM deficient quails resulted in only local correction (Tsujino et al., 1998). The failure to provide a systemic correction was attributed to a relatively short duration of the transgene expression. However, it is possible that even if expressed for relatively long periods, enzyme production in vivo through muscle gene therapy may not be effective for improving function of distant organs.

Recent in-vivo studies have demonstrated the benefits of hepatic-targeted transduction for gene therapy of GSDII (Amalfitano et al., 1999.; Pauly et al., 2001). However, there remains a need for methods of treating GM-deficiency disorders, as well as other disorders resulting from lysosomal enzyme deficiencies, which do not involve in vivo gene therapy. This need arises from safety concerns regarding in vivo gene therapy, and from a desire to provide careful regulation of the amount of replacement enzyme administered.

The current invention provides methods for producing lysosomal enzymes, including GAA, and protein products of these methods. The protein products are effective for in vivo treatment of disorders involving deficiencies in the enzymes. The methods allow effective regulation of the amount of lysosomal enzyme administered.

### SUMMARY OF THE INVENTION

The invention generally relates to the production of native recombinant lysosomal enzymes by liver cells, preferably in vitro, and methods of using the native recombinant lysosomal enzymes to treat enzyme deficiencies in vivo. Lysosomal enzymes, including acid alphaglucosidase (GM), produced by liver cells apparently undergo the post-translational modifications necessary to achieve good enzymatic activity. The resulting enzymes can be taken up by various other cells and can correct phenotypic abnormalities of distant organs with enzyme deficiencies. In certain preferred embodiments, the enzymes and methods are especially adapted for treatment of type II glycogen storage disease in mammals, including humans.

In one aspect, the current invention provides a method for preparing a lysosomal enzyme suitable for replacement therapy. In the method, an expression vector is provided that is capable of expressing the enzyme in liver cells. The expression vector is then introduced into liver cells, wherein the lysosomal enzyme is expressed. The expressed lysosomal enzyme is then collected. In preferred embodiments, the lysosomal enzyme is acid alpha-glucosidase. Preferably, the expressing step is performed in vitro using cultured liver cells. Most preferably, both the expressing step and the introducing step are performed in vitro using cultured liver cells.

In another aspect, the current invention is a lysosomal enzyme suitable for replacement therapy, produced by the above-described method. Preferably, the lysosomal enzyme is acid alpha-glucosidase.

In another aspect, the current invention is a method for treating a deficiency of a lysosomal enzyme in a mammal by introducing an effective amount of the lysosomal enzyme made as described above, into the mammal. Preferably, the lysosomal enzyme is acid alpha-glucosidase, and the mammal is a human.

In another aspect, the current invention provides cultured mammalian liver cells transformed with an expression vector capable of expressing a lysosomal enzyme. Preferably, the lysosomal enzyme is acid alpha-glutaminase.

In another aspect, the current invention provides a method for treating a deficiency of a lysosomal enzyme in a mammal by introducing an expression vector that expresses the lysosomal enzyme into the liver of the mammal. Preferably, expression of the lysosomal enzyme is regulated by a regulatory factor and the lysosomal enzyme is acid alpha-glucosidase. In certain embodiments, the regulatory factor is provided to the mammal by oral administration. In certain preferred embodiments, the regulatory factor is an antibiotic, for example, tetracycline.

### BRIEF DESCRIPTION OF THE FIGURES

FIG. 1. Expression of GFP in skeletal muscle of Mck-T-hGAA/−/− mice. Each of the three founder lines containing human GAA cDNA-IRES-GFP (F.06, F.53, and F.17) were crossed with Mck-T/−/− mice to give rise to the three double transgenic lines expressing different levels of hGAA as reflected by the difference in the GFP expression ranging from low in F.06 to high in F.17. Muscle cells were examined under a fluorescent microscope using excitation light.

FIG. 2. Detection of hGAA protein in the low expresser line (F.06) by Western blot analysis: spleen (S), liver (Li), heart (H), skeletal muscle (M). The 76 kDa processed form of hGAA is detected only in skeletal muscle lysates from double transgenic mice. Control-tissue lysates are from age-matched single transgenic (Mck-T/−/−) animal. The gels were loaded with 100 μp protein; immunodetection of vinculin was used for loading control.

FIG. 3. Analysis by light microscopy (PAS-stained sections) of skeletal muscle (Panel A); heart, liver, and brain from single transgenic (Mck-T/−/−) control (Panel B); and the low expresser line (Panel C) at 4 months of age. Original magnification: X20 except for the top right panel which is X40.

FIG. 4. Detection of hGAA mRNA and protein in the intermediate expresser line Mck-T-hGAA/−/− (F.53). Panel A: Northern blot analysis. Total RNA from respective tissues was analyzed as described in the Examples: M—skeletal muscle; H—heart; Li—liver; Lu—lung; B—brain; K—kidney; S—spleen. The transgene is expressed only in skeletal muscle and heart. Lower panel shows ethidium bromide stained gel used for Northern blot. Panel B: RT-PCR analysis detected a 170 bp product only in skeletal muscle and heart. Panel C: Immunodetection of hGAA (upper panel) and GFP (lower panel) proteins using specific antibodies. Note that the gel was loaded with 10 μg of protein for skeletal muscle and heart. hGAA is a 76 kDa protein; GFP is a 29 kDa protein. hGAA protein is detected not only in skeletal muscle and heart but also in liver (Li), lung (Lu), and spleen (S); 110 kDa precursor form is detected in plasma (P) after a prolonged exposure.

FIG. 5. Analysis by light microscopy (PAS-stained sections) of skeletal muscle, liver, and brain from the intermediate expresser line at 5 months of age. Original magnification: X20 for muscle and liver; X40 for brain.

FIG. 6. Analysis by light microscopy (PAS-stained sections) of skeletal muscle and heart from the high expresser line at 5 months of age. Panel A: original magnification X40; Panel B: original magnification X63.

FIG. 7. Expression of GFP in livers of the Alb-T-hGAA/−/− mice. Liver cells were examined under a fluorescent microscope using excitation light. Two double transgenic lines derived from two independent founders (F. 21 and F. 26) showed similar levels of GFP expression.

FIG. 8. Detection of hGAA mRNA and protein in the Alb-T-hGAA line. Panel A: Northern blot analysis. Total RNA from liver (Li), muscle (M), and heart (H). The hGAA transgene is expressed only in liver. Lower panel shows ethidium bromide stained gel used for Northern blot. Panel B: RT-PCR analysis of cDNA from respective tissues. The primers in exon 5 and 7 of the human GM detected a 270 bp product only in liver (top); Southern blot analysis with a probe in exon 6 detected a signal only in liver. Panel C: Immunodetection of hGAA (upper panel) and GFP (lower panel) proteins using specific antibodies. Note that the gel was loaded with 1 Opg of protein for liver. hGAA is a 76 kDa protein; GFP is a 29 kDa protein. hGAA protein is detected not only in liver, but also in muscle (M) and heart (H); 110 kDa precursor form is easily detectable in plasma (P). Single transgenic (Alb-T/−/−) animal was used as a control.

FIG. 9. Analysis by light microscopy (PAS-stained sections) of liver, muscle, and heart from single transgenic control (AlbT/−/−) and double transgenic Alb-T-hGAA/−/− mice at 3 months of age. Original magnification was X20.

### DETAILED DESCRIPTION OF INVENTION

The invention generally relates to the production of native recombinant lysosomal enzymes by liver cells, preferably in vitro. Lysosomal enzymes, including acid alpha-glucosidase (GAA), produced by liver cells apparently undergo post-translational modifications to achieve good enzymatic activity. The resulting lysosomal enzymes can be taken up by various other cells and can correct phenotypic abnormalities of distant organs caused by enzyme deficiencies. Thus, lysosomal enzymes produced by the liver cells are effectively or well processed.

In one aspect, the current invention provides a method for preparing a lysosomal enzyme suitable for replacement therapy. In the method, an expression vector is provided that is capable of expressing the lysosomal enzyme in liver cells. Expression vectors that are capable of expressing inserted proteins in liver cells in vivo and in vitro are well known in the art. Expression vectors include heterologous nucleic acid sequences, that is nucleic acid sequences that are not naturally found adjacent to nucleic acid molecules of the present invention and that preferably are derived from a species other than the species from which the nucleic acid molecule(s) are derived.

The vector can be either RNA or DNA, either prokaryotic or eukaryotic, and typically is a virus or a plasmid. One type of recombinant vector, referred to herein as a lysosomal enzyme expression vector, comprises a nucleic acid molecule encoding a lysosomal enzyme, operatively linked to an expression vector. The phrase “operatively linked” refers to insertion of a nucleic acid molecule into an expression vector in a manner such that the molecule is expressed when transformed into a host cell.

Primary liver cell cultures can be established from cells isolated directly from mammalian livers, including human livers, as is known in the art. Alternatively, a number of liver cell lines have been established that may be used with the current invention. Liver cell lines may be obtained, for example, from the American Type Culture Collection (Manasuss, Va.). These liver cell lines include, but are not limited to, SNU-398, SNU-387, and SNU-423, all derived from anaplastic hepatocellular carcinomas. Additional liver cell lines suitable for use in this invention can be prepared using standard techniques well know in the art.

As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in mammalian liver cells.

Expression vectors of the present invention may contain one or more regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences, which control the initiation, elongation, and termination of transcription. Particularly important transcription control sequences are promoter sequences.

Suitable transcription control sequences include any transcription control sequence that can function in at least one of the recombinant cells of the present invention. A variety of such transcription control sequences are known to those skilled in the art. Examples of control sequences include, but are not limited to, metallothionein, alpha-mating factor, Pichia alcohol oxidase, alphavirus subgenomic promoter, antibiotic resistance gene, baculovirus, Heliothis zea insect virus, vaccinia virus, herpesvirus, raccoon poxvirus, other poxvirus, adenovirus, cytomegalovirus (such as immediate early promoter), simian virus 40, retrovirus, actin, retroviral long terminal repeat, Rous sarcoma virus, heat shock, phosphate and nitrate transcription control sequences as well as other sequences capable of controlling gene expression in mammalian liver cells.

Preferred transcription control sequences are liver-specific promoters and enhancers. For example, a preferred promoter is the albumin liver-specific promoter, as illustrated in the Examples section of this specification. Transcription control sequences of the present invention can also include naturally occurring transcription control sequences naturally associated with a mammalian lysosomal enzyme gene, such as human GM gene transcription control sequences.

Recombinant molecules of the present invention typically contain secretory signals (i.e., signal segment nucleic acid sequences) to enable an expressed liver enzyme of the present invention to be secreted from the cell that produces the protein and/or fusion sequences which lead to the expression of nucleic acid molecules of the present invention as fusion proteins. Examples of suitable signal segments include any signal segment capable of directing the secretion of a protein of the present invention. Preferred signal segments include, but are not limited to, tissue plasminogen activator (t-PA), interferon, interleukin, growth hormone, histocompatibility and viral envelope glycoprotein signal segments.

In certain examples, genes of lysosomal enzymes in their native form include a signal segment nucleic acid followed by a segment encoding a mature lysosomal enzyme. Therefore, for these lysosomal enzymes, an endogenous signal segment is preferably included, rather than an exogenous segment. Eukaryotic recombinant molecules can also include intervening and/or untranslated sequences surrounding and/or within the nucleic acid sequences of nucleic acid molecules of the present invention.

Host liver cells can be either untransformed cells or cells that are already transformed with at least one nucleic acid molecule. A recombinant liver cell is preferably produced by transforming a host liver cell with one or more recombinant molecules, each comprising one or more nucleic acid molecules of the present invention operatively linked to an expression vector containing one or more transcription control sequences. A recombinant cell of the present invention includes any cell transformed with at least one of any nucleic acid molecule of the present invention. Suitable and preferred nucleic acid molecules, as well as suitable and preferred recombinant molecules with which to transfer cells, are disclosed herein.

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control is signals (e.g., ribosome binding sites, Shine-Dalgamo sequences), modification of nucleic acid molecules of the present invention to correspond to the codon usage of the host cell, deletion of sequences that destabilize transcripts, and use of control signals that temporally separate recombinant cell growth from recombinant enzyme production during fermentation.

Transformation of a nucleic acid molecule, such as a recombinant lysosomal enzyme-expressing vector, into a liver cell can be accomplished by any method by which a nucleic acid molecule can be inserted into the cell.

Transformation techniques include, but are not limited to, transfection, infection, electroporation, microinjection, lipofection, adsorption, and protoplast fusion. Transformation may be stable or transient. It is to be noted that a cell line refers to any immortalized recombinant cell of the present invention that is not a transgenic animal. Transformed nucleic acid molecules of the present invention can remain extrachromosomal or can integrate into one or more sites within a chromosome of the transformed (i.e., recombinant) cell in such a manner that their ability to be expressed is retained.

In embodiments where the recombinant lysosomal enzyme is produced in vivo, the recombinant lysosomal enzyme is typically collected by initially isolating the liver or other tissue in which the recombinant protein is expressed, and isolating proteins from the tissue using standard techniques. The lysosomal enzyme may then be purified using standard techniques.

Virtually any lysosomal enzyme may be produced in liver cells using the methods of the current invention. Lysosomal enzymes will benefit from the current invention because they typically involve extensive post-translational processing, as described above. Lysosomal enzymes for use with the current invention include, but are not limited to, acid alpha-glucosidase, and the like. In preferred embodiments, the lysosomal enzyme is an enzyme in which mannose 6-phosphate moieties are attached post-translationally. Most preferably, the lysosomal enzyme is acid alpha-glucosidase.

Preferably, the expressing step is performed in vitro using cultured liver cells. Most preferably, both the expressing step and the introducing step are performed in vitro using cultured liver cells, as described above.

The phrase “collecting the lysosomal enzyme”, as well as similar phrases, refers to collecting the whole medium containing the recombinantly-produced lysosomal enzyme and need not imply additional steps of separation or purification. Lysosomal enzymes of the present invention can be purified, if desired, using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing, and differential solubilization. Lysosomal polypeptides of the present invention are preferably retrieved in “substantially pure” form. As used herein, “substantially pure” refers to a purity that allows for the effective use of the protein as a therapeutic composition or diagnostic. A therapeutic composition for animals, for example, should exhibit no substantial toxicity and preferably should be capable of being taken up by cells that are affected by the enzyme deficiency.

The term “isolated lysosomal enzyme” as used in this specification, refers to the removal of a lysosomal enzyme from its natural environment and does not describe any specific level of purity of the polypeptide.

In certain embodiments, the isolated lysosomal enzymes of the current invention include additional purification fusion polypeptide segments that assist in purification of the lysosomal enzymes. A suitable purification fusion segment can be a domain of any size that plays a role in purification of the fusion polypeptide. Purification fusion segments can be joined to the amino and/or carboxyl terminus, and can be susceptible to cleavage in order to enable recovery of the lysosomal enzyme.

Recombinant lysosomal enzymes of the current invention containing purification fusion segments are preferably produced by culturing a recombinant cell transformed with a polynucleotide capable of expressing lysosomal enzymes. A number of purification fusion segments have been described. For example, fusion segments include metal binding domains (e.g., poly-histidine segment), immunoglobulin binding domains (e.g., Protein A; Protein G; T cell; B cell; Fc receptor or complement protein antibody-binding domains), sugar binding domains (e.g., maltose binding domain), and/or a “tag” domain (e.g., at least a portion of β-alactosidase, strep tag peptide, T7 tag peptide, Flag peptide, or other domains that can be purified using compounds that bind to the domain, such as monoclonal antibodies).

In another aspect, the current invention is a lysosomal enzyme suitable for replacement therapy, produced by the above-described method. Preferably, the lysosomal enzyme is acid alpha-glucosidase.

In another aspect, the current invention is a method for treating a deficiency of a lysosomal enzyme in a mammal by introducing an effective amount of the lysosomal enzyme made as described above into the mammal. Preferably, the lysosomal enzyme is acid alpha-glucosidase, and the mammal is a human. Typically, for these embodiments, the lysosomal enzyme is in admixture with a pharmaceutically acceptable carrier or diluent.

The term “effective amount” of the lysosomal enzyme means an amount effective at dosages and for periods of time necessary to provide lysosomal enzymes to enzyme-deficient cells. Preferably, the effective amount is an amount that at least partially corrects cellular functional defects that result from decreased lysosomal enzyme levels.

For the lysosomal enzymes of the current invention, standard techniques can be used to determine an effective amount. In particular, an effective amount may be determined by techniques well-known to those skilled in the medical or veterinary arts taking into consideration such factors as the condition of the animal intended for administration (i.e., weight, age, and general health of the animal), the mode of administration, and the type of formulation. The amount of the lysosomal enzyme as well as its dosage regime may be adjusted to provide the optimum replacement of enzyme activity. Typically, a determination of an effective amount of the lysosomal enzyme involves an analysis of toxicity. Toxicity may be analyzed by determining a lethal dose (LD) and LD₅₀ in a suitable animal model (e.g., a mouse model).

The lysosomal enzymes of the current invention may be administered by any conventional route. Conventional routes include, for example, mucosal (e.g., ocular, intranasal, oral, gastric, pulmonary, intestinal, rectal, vaginal, or urinary tract), parenteral (e.g., subcutaneous, intradermal, intramuscular, intravenous, or intraperitoneal), or intranodal routes. The administration may be by injection, oral administration, inhalation, transdermal application, rectal administration, or any other route that enables replacement of deficient enzyme function. In certain preferred embodiments, the administration is by intravenous injection. The administration can be achieved in a single dose or repeated at intervals.

The term “pharmaceutically acceptable” means approved by a regulatory agency of the Federal or a state government of the U.S or other countries or listed in the U.S. Pharmacopeia or other generally recognized pharmacopeia for use in animals, and more particularly-in humans. The term “carrier” refers to a diluent, adjuvant, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents and/or pH buffering agents. These compositions can take the form of solutions, suspensions, emulsion, tablets, pills, capsules, powders, sustained-release formulations and the like. The compositions can be formulated as suppositories with traditional binders and carriers such as triglycerides. Oral formulations can include standard carriers such as pharmaceutical grades of mannitol, lactose, starch, magnesium stearate, sodium saccharine, cellulose, magnesium carbonate, and the like. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients may be mixed prior to administration.

In another aspect, the current invention provides cultured mammalian liver cells transformed with an expression vector capable of expressing a lysosomal enzyme. Preferably, the lysosomal enzyme is acid alpha-glutaminase. Culturing conditions, cell types, and the like are described above.

In another aspect, the current invention provides a method for treating a deficiency of a lysosomal enzyme in a mammal by introducing into the liver of the mammal, an expression vector that expresses the lysosomal enzyme. Preferably, expression of the lysosomal enzyme is regulated by a regulatory factor and the lysosomal enzyme is acid alpha-glucosidase. In certain embodiments, the regulatory factor is provided to the mammal by oral administration. In certain preferred embodiments, the regulatory factor is an antibiotic, for example, tetracycline.

In another aspect, the current invention provides a cultured mammalian liver cell transformed with an expression vector capable of expressing a lysosomal enzyme. In certain preferred embodiments, the lysosomal enzyme is acid alpha-glutaminase.

The following examples describe and illustrate the processes and products of the invention. These examples are intended to be merely illustrative of the present invention, and not limiting thereof in either scope or spirit. Unless indicated otherwise, all percentages and ratios are by weight. Those skilled in the art will readily understand that variations of the materials, conditions, and processes described in these examples can be used. All references cited herein are incorporated by reference.

### EXAMPLE 1

This example compares the ability of constitutive expression of GM in muscle cells versus liver cells to correct defects that result from insufficient expression of GM. Generation of GM knockout mice has been reported previously; the knockout mice developed a generalized glycogen storage deficiency, resembling both the human severe infantile and milder forms of the disease (Raben et al., 1998; Raben et al., 2000).

Generation of Transgenic Mouse Strains. (1). A transgenic mouse strain containing human GM cDNA (hGAA) under the control of a tetracycline responsive element (TRE) (pTRE-hGAA) was established as follows: For construction of the pTRE-hGAA transgene, human GM cDNA was released from pCDNA3 (a kind gift from Dr. Frank Martinuk, NYU) with HindIIIXhoI digestion, and the fragment was cloned into HindIII/SaII sites of pTRE2 to give rise to pTRE2-hGAA (Clontech, Palo Alto, Calif.). In this plasmid, the hGAA is linked to the CMV minimal promoter fused to the tetracycline responsive element (TRE). Next, the IRES-EGFP fragment was released from pIRES2-EGFP (Clontech, Palo Alto, Calif.) by digestion with SmalI/Xbal digest, and cloned into pTRE2-hGAA to give rise to pTRE2-hGAA-IRES-EGFP. The TRE-hGAA-IRES-EGFP-B-globin poly A fragment was released by XhoI/SapI digest, gel purified by electroelution, and used for microinjection into FVB embryos. These mice are referred to as hGAA.

(2). A transgenic mouse strain containing the tetracycline-controlled transcriptional activator (tTA) under the control of liver specific albumin promoter was established as follows. For construction of the Alb-tTA transgene, the tTA fragment linked to SV40 poly A was cloned into the PstI sites of pcDNA2.1 (Invitrogen, San Diego, Calif.) to give rise to pcDNA2.1-tTA. Next, the mouse albumin promoter/enhancer was released from pGEMAlbSVPA (a kind gift from Dr. T. Jake Liang, NIDDK, NIH) with ApaI/NotI digest and cloned into pcDNA2.1-tTA to generate pcDNA2.1-Alb-tTA (the NotI site was destroyed during cloning). Then, a 500 bp fragment of the chicken B-globin core insulator (Recillas-Targa et al. 1999) was PCR amplified from pNI-CD (a kind gift from Dr. Gary Felsenfeld, NIDDK, NIH) and cloned into both ApaI and SapI sites of the pcDNA2.1-tTA. The insulators flank the Alb-tTA sequence within pcDNA2.1. The insulator-Alb-tTA-SV40 poly A-insulator fragment was released by PacI digestion (PacI sites were introduced during PCR amplification), gel purified by electroelution, and used for microinjection into FVB embryos. These mice are referred to as Alb-T.

(3). A transgenic mouse strain containing tetracycycline-controlled transcriptional activator (tTA) under the control of muscle specific creatine kinase promoter (Mck-tTA) was also employed. These mice are referred to as Mck-T.

The tissue-specific hGAA expression in the knockout mice was accomplished by using either the liver-specific mouse albumin promoter, or the muscle-specific creatine kinase promoter to drive the tetracycline-senstive transactivator. A control line contained the human GM cDNA under the control of the tetracycline responsive element to which the transactivator binds in the absence of tetracycline.

Genotyping of Transgenic Lines and Breeding Strategy. Southern analysis of the founders and F1 progeny from both hGAA and Alb-T transgenic lines revealed integration of the injection fragment. Genomic DNA was isolated from tail clips and genotyping of the transgenic mice was performed using standard procedures. For hGAA, a 733 bp fragment from pIRES2-EGFP vector (BstxI/XbaI digest) was used as a probe after digestion of tail DNA with EcoRI, resulting in an about 2 kb fragment from Southern analysis. For Mck-T and Alb-T, an about 1.5 kb fragment from pTet-Off (Clontech, Palo Alto, Calif.) (EcoRI/HindIII digest) was used as a probe after digestion of tail DNA with EcoRI, resulting in about 13 kb and about 2.6 kb fragments from Southern analysis, respectively. All probes were labeled by the random hexamer method (Lofstrand, Gaithersburg, Md.). Each of the three transgenic strains were crossed to the knockout mice in which the acid alpha-glucosidase gene (GAA) was disrupted by a neo cassette placed in the middle of exon 6 (Raben et al., 1998). The knockout mice (GAA−/−) were crossed to each of the two transgenic mouse lines. Tail DNA was screened by Southern analysis to identify GAA−/− mice, as described (Raben et al., 1998). The resulting lines are referred to as hGAA/−/−, Mck-T/−/−, and Alb-T/−/−.

Mck-T/−/− mice were mated to hGAA/−/− mice to generate double transgenic mice expressing hGAA in skeletal muscle of the knockout mice (C57BL6/129 SVj), referred to as Mck-T-hGAA/−/−. The Alb-T/−/− mice were also mated to hGAA/−/− to generate double transgenic mice expressing hGAA in liver of the knockout mice (FVB×C57BL6/129 SVj), Alb-T-hGAA/−/−.

Administration of doxycycline (dox). Slow release doxycycline pellets (Innovative Research of America, Sarasota, Fla.) were implanted s.c. using a trochar as described by the manufacturer. These 90-day release pellets delivered about 0.7 mg doxycycline hydrophloride per day. Alternatively, mice on dox treatment were given 2 mglml dox in a 5% sucrose solution instead of normal drinking water. Dox solution was maintained in dark bottles and changed twice a week. Females were administered dox pellets prior to mating so that the transgenic offspring were exposed to the drug in utero.

Light microscopy. Tissues were fixed in 10% formalin, embedded in paraffin, and stained in hematoxilineosin or periodic acid-Shiff (PAS) by standard methods. The animals were starved overnight before sacrifice. For GFP detection, tissues were snap frozen and embedded in paraffin. Frozen sections were examined under a fluorescent microscope.

GAA enzyme assay, Western blot analysis and Glycogen content. GM activity in the tissue homogenates was measured by conversion of the substrate 4-methylumbelliferyl (4-MU) α-D-glucoside to the fluorescent product umbelliferone as described (Hermans et al., 1991). Blood spots on Guthrie cards were used to determine GAA activity in plasma as described previously (Umapathysivam et al., 2000).

Tissues were homogenized in lysis buffer (300 mM NaCl, 50 mM Tris, 2 mM EDTA, 0.5% Triton X-100) with proteinase inhibitors (4 mM Pefabloc SC, 10 μg/ml aprotinin, 10 μg/ml leupeptin). Homogenates were centrifuged at 15,000×g for 30 min at 4° C. and the supernatants were collected. Total protein concentration of the lysates were measured using Bio-Rad protein assay reagent (Bio-Rad Laboratores, Hercules, Calif.). Samples were electrophoresed on SDS-PAGE gels and electrotransfered to nitrocellulose. The blots were incubated with rabbit antiserum to human GAA (a kind gift of Y.T. Chen, Duke University). Immunodetection was performed with goat anti-rabbit-IgG conjugated to horseradish peroxidase in combination with chemiluminescence (Santa Cruz Biotechnology, Santa Cruz, Calif.). Living Colors A.v. monoclonal antibody (Clontech, Palo Alto, Calif.) and anti-mouse-IgG-HRP (Santa Cruz Biotechnology, Santa Cruz, Calif.) were used to detect EGFP activity.

Glycogen concentration was determined by measuring the amount of glucose released after treatment of tissue extracts with Aspergillus niger amyloglucosidase as previously described (Kikuchi et al., 1998).

Northern analysis and RT-PCR. Total RNA was isolated from tissues using TRizol Reagent (Life Technologies, Gaithersburg, Md.): Northern analysis was performed according to standard procedure using an about 1.2 kb SmaI fragment of the hGAA as a probe.

Total RNA was digested with DNasel using a DNA-free kit (Ambion, Austin, Tex.) as recommended by the manufacturer. First strand cDNA was primed from 1 μg RNA with random hexamers according to the manufacturer's instructions (Boehringer Mannheim, Indianapolis, Ind.). cDNA samples (2 μl) were used for PCR amplifications with primer pair cctttctacctggcgctggaggac/ggtgatagcggtggaggagta (human GAA exon-5-sense and exon-7-antisense) or cccttcatgcggaaccacaacagcctgctc/agaggggccgggccacggtctcccccgc (human GAA exon-14-sense and exon-15-antisense). PCR was performed using SuperMix (Life Technologies, Gaithersgurg, Md.). Conditions were denaturation at 95° C. for 5 min., followed by 35 cycles at 95° C. for 1 min., annealing at 55° C. for 1 min., and extension at 72° C. for 1 min.

Statistical analysis. Data in text and figures are given as mean ±S.E. The Student's t test was used for comparisons between the groups. Differences were considered significant at p<0.005.

Expression of hGAA in skeletal muscle of the knockout mice. Three transgenic hGAA founder lines (F.06, F.53, and F.17) were generated; each was crossed to GAA knockout mice (−/−), resulting in three hGAA/−/− lines. The transgene also contained a sequence coding for green fluorescent protein (GFP) which was used to assess the expression pattern and effectiveness of this conditional system.

Each of these lines, in turn, was crossed to Mck-T transgenic mice, already on a knockout background (Mck-T/−/−), to generate double positive for the experiment. Double transgenic mice, Mck-T-hGAA/−/−, were designed to constitutively express hGAA in skeletal muscle of these knockout animals. Single positive mice carrying either hGAA or Mck-T on either heterozygous (±) or homozygous (−/−) knockout background were used as controls for the experiment.

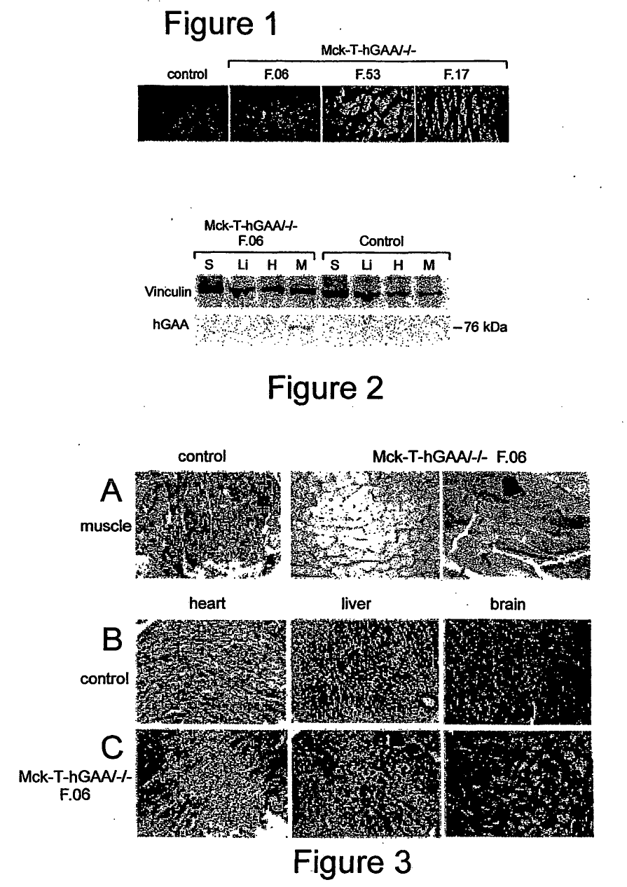

Single transgenic mice on a knockout background (−/−) showed a very low, probably non-specific enzyme activity in all tissues tested (Table 1). The double transgenic lines derived from each of the three hGAA founders expressed low, intermediate, and high levels of hGAA in the skeletal muscle as reflected by the intensity of GFP (FIG. 1).

In the low expresser line, F.06, the level of hGAA activity in skeletal muscle exceeded the levels in ± mice by about 5 times as shown in Table 1.

TABLE 1
GAA activity in double transgenic mice and single transgenic
controls (nmol/h/mg protein).

| Founder | Muscle | Heart | Liver | Lung | Brain | Spleen | Plasma |
|---|---|---|---|---|---|---|---|
| Mck-T- | 28.9 ± 4.9 | 1.4 ± 0.4 | 3.4 ± 0.7 | 2.6 ± 0.9 | 0.5 ± 0.4 | 2.8 ± 0.7 | 0 |
| hGAA/-/- | | | | | | | |
| F.06 n = 8 | | | | | | | |
| Mck-T- | 466 ± 90 | 223 ± 64 | 13 ± 1.9 | 4.0 ± 1.96 | 0.26 ± 0.2 | 12.8 ± 2.5 | 4.0 ± 0.7 |
| hGAA/-/- | | | | | | | |
| F.53 n = 5 | | | | | | | |
| Control | 1.9 ± 0.6 | 0.9 ± 0.4 | 2.3 ± 0.4 | 1.2 ± 0.4 | 1.45 ± 0.8 | 2.0 ± 0.6 | 0 |
| (−/−)* | | | | | | | |
| n = 9 | | | | | | | |
| Control | 5.6 ± 0.5 | 9.0 ± 0.7 | 26 ± 3.0 | 5.8 ± 1.5 | 39.4 ± 6.6 | 16.0 | — |
| (+/−)* | | | | | | | |
| n = 11 | | | | | | | |
| Alb-T- | 27.8 ± 5.5 | 35 ± 9.0 | 659 ± 47 | — | 1.5 ± 0.4 | — | 424 ± 62 |
| hGAA/-/- | | | | | | | |
| n = 8 | | | | | | | |

*Single transgenics (Mck-T, hGAA, and Alb-T) on GAA homozygous (−/−) or heterozygous background (+/−) were used as controls.

GAA protein was detected by western analysis in skeletal muscle, but not any other organ (FIG. 2). The increase of enzyme activity in skeletal muscle was accompanied by a normalization of glycogen level to wild type values as shown in Table 2.

TABLE 2
Glycogen content in double transgenic and control mice (% wet weight).

| | | Heart | | | |
|---|---|---|---|---|---|
| Genotype/ | | 5-8 | 4-8 | | |
| Founder | Muscle* | weeks | months | Diaphragm | Tongue |
| Mck-T-hGAA/-/- | 0.17 ± 0.05 | 1.6 ± 0.6 | 8.9 ± 2.2 | — | 5.2 ± 0.12 |
| F.06 | n = 5 | n = 3 | n = 5 | | n = 2 |
| Mck-T-hGAA/-/- | 0.33 ± 0.08 | — | 0.13 ± 0.07 | 0 | — |
| F.53 | n = 8 | | n = 5 | n = 4 | |
| Control (−/−) | 3.2 ± 0.5 | 1.5 ± 0.4 | 7.2 ± 1.4 | 3.7 ± 0.6 | 5.16 ± 0.15 |
| | n = 7 | n = 2 | n = 5 | n = 3 | n = 2 |
| Alb-T-hGAA/-/- | 0.3 ± 0.1 | 0.18 ± 0.09 | 0.15 ± 0.04 | — | — |
| | n = 6 | n = 4 | n = 2 | | |
| wt [n = 7]** | 0.17 ± 0.08 | ND | ND | 0.03 ± 0.002 | — |

*double transgenics [6 w-8 m]; controls [3 m-8 m]
**previously reported data (Raben at al. 2000)

Accordingly, light microscopy showed the phenotypic correction of skeletal muscle, with the majority of the cells cleared from glycogen (FIG. 3, Panel A). However, the phenotypic correction in skeletal muscle was not associated with any detectable enzyme activity in plasma (Table 1), nor was there any significant increase in the levels of GAA activity in the distant organs when compared to background levels seen in knockout mice. Glycogen level in the heart progressively increased, and the rate of increase was similar to that in the knockouts. Thus, no secretion or uptake occurred with this level of gene expression in skeletal muscle, as supported by the accumulation of glycogen in heart, liver, and brain, detected by light microscopy (FIG. 3, Panel C).

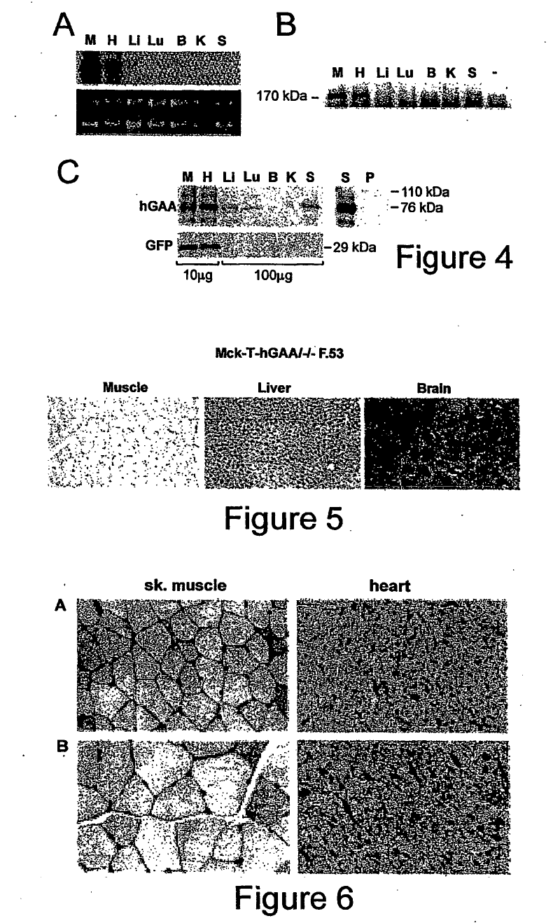

In the intermediate expresser lines, the level of hGAA activity in skeletal muscle exceeded the level in ± mice by about 80 times (Table 1). As shown by northern analysis and RT-PCR, hGAA was expressed in both skeletal muscle and heart (FIG. 4, Panels A and B), but not in liver, lung, brain, kidney, or spleen of the double transgenic knockout mice, providing target organs to study systemic correction. The lack of GFP expression in these target organs by western analysis further supported the RT-PCR data (FIG. 4, Panel C). The hGAA protein, however, was detected in most of the target organs—liver, lung, and spleen (FIG. 4, Panel A), suggesting that the enzyme was secreted by skeletal muscle and up-taken. Increased GAA activity was detected in liver, lung, and spleen, and reached about 50%, 69%, and 80% of values in heterozygous controls, respectively (Table 1). To confirm that the GAA activity in these organs was the result of cross-correction by circulating enzyme, plasma was analyzed from double transgenic mice for the presence of hGAA. Low level of GAA activity could be detected in plasma by the enzyme assay (Table 1) and western analysis; the 110 kDa precursor protein was visible after prolonged exposure (FIG. 4, Panel C). Morphologically, skeletal muscle was completely cleared of glycogen and the secreted protein provided a phenotypic rescue in distant organs (shown for liver cells in FIG. 5). No protein was detected in brain and kidney (FIG. 4, Panel C), and morphologically, no improvement was observed in these organs (shown for brain in FIG. 5).

In the high expresser line, hGAA was expressed in skeletal muscle and heart, and the levels of enzyme activity reached 1082±142 and 793±85 nmol/h/mh/protein, respectively. These levels exceeded those in ±mice by about 200 times in skeletal muscle and by about 90 times in heart. Not only did these levels fail to efficiently correct the phenotype of the knockout animals, they might be toxic to the cells. Both skeletal and cardiac muscles contained multiple PAS positive cells, with some skeletal muscle cells of abnormal shape and smaller size (FIG. 6). For this reason, this line was not further analyzed.

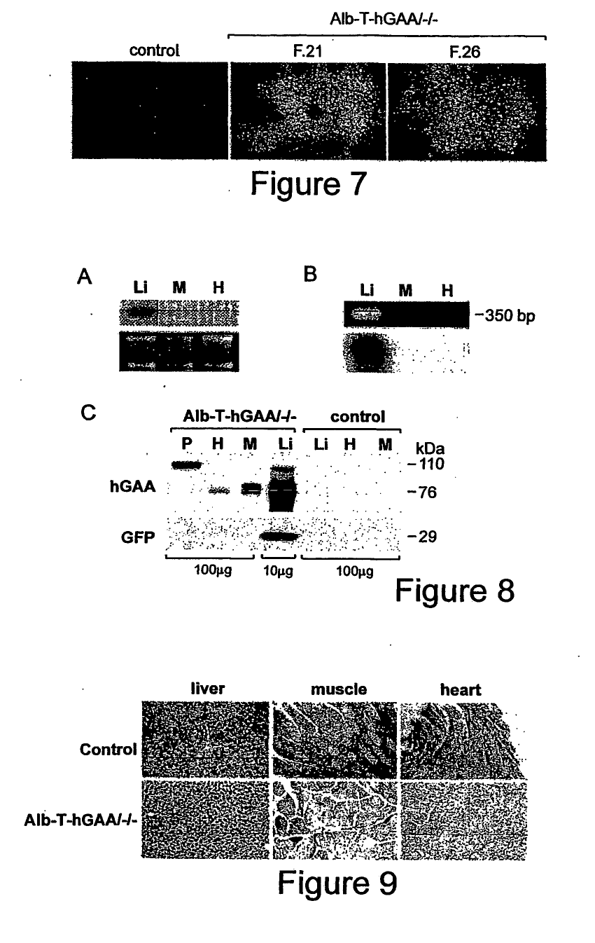

Expression of hGAA in liver of the knockout mice. Two transgenic Alb-T founder lines (F.21 and F.26) were generated and each of them was crossed to GAA knockout mice, resulting in two Alb-T/−/− lines. Both were then crossed to hGAA/−/− (F. 17) to generate double positive mice that constitutively express hGAA in livers of these knockout mice. Single positive transgenic mice carrying Alb-T on either heterozygous (±) or homozygous (−/−) knockout background were used as controls for the experiment. The expression of the transgene in livers of double transgenic mice was similar in the two lines, as shown by the intensity of GFP staining (FIG. 7), and by the levels of hepatic GAA activity exceeding about 25 times those in heterozygous controls. Thus, only one line (F.21/F.17) is presented in the study.

hGAA expression was detected only in the liver in double transgenic mice. Northern analysis and RT-PCR for hGAA mRNA revealed a complete absence of the transgene expression in skeletal muscle and heart—the two most affected organs of GSDII (FIG. 8, Panels A and B). However, hGAA protein was detected in all three organs by western analysis, supporting the concept that the enzyme was secreted by liver and taken up by the skeletal muscle and heart. Indeed, very high levels of 110 kDa precursor protein was detected in plasma by western analysis. The level of enzyme activity in plasma was extremely high (424 nmol/h/mg)—more than 100 times greater than the values observed in the Mck-T-hGAA/−/− mice. Both skeletal muscle and heart were sensitive to cross-correction by circulating enzyme; GAA activity in these organs was significantly elevated compared to the levels in heterozygous controls—about 5-fold increase in skeletal muscle and about 4-fold increase in heart (Table 1). Light microscopy showed that the high level of hGAA expression seemed to be well tolerated by the liver, with most of the cells cleared of glycogen. Both skeletal muscle and heart appeared morphologically normal, with the exception of occasional muscle fibers, where lysosomal accumulation of glycogen was observed (FIG. 9). Consistent with these results, the level of glycogen in skeletal muscle and heart were at or near undetectable levels, 0.33% and 0.18%, respectively (Table 2). Thus, the systemic correction of the phenotype in the knockout mice is achieved much more efficiently when the hGAA gene is expressed in liver, compared to skeletal muscle.

Regulation of expression by tetracycline. The intermediate expresser line of Mck-T/hGAA/−/− double transgenic mice was used for doxycycline experiments. Littermates were divided into two groups, one which remained on doxycycline since birth (gene off group) and the other group in which doxycycline was removed after 3 weeks (gene off/on group). Three weeks on doxycycline resulted in complete inactivation of the hGAA enzyme in skeletal muscle and accumulation of glycogen in muscle and heart similar to that in the single transgenic age matched controls. The dox treatment did not affect the single transgenic control mice. Mice from the gene off/on group were sacrificed after doxycycline was removed for five weeks.

Results of the present experiment indicate that even when most of the muscles in the body constitutively express about 5 times more enzyme (low expresser line) than heterozygous controls, no secretion and no uptake occurs. Interestingly, occasional muscle cells in this line still contain lysosomal glycogen, indicating that this level of enzyme does not provide the correction of even neighboring cells. Since the expression of the transgene in this line is limited to skeletal muscle, other internal organ including heart and brain, accumulate glycogen at a rate similar to that in the knockout animals.

A low level of secretion was observed in mice constitutively expressing about 80-fold increased levels of GAA activity (compared to±controls) in skeletal muscle (intermediate expresser line). The high GAA levels in the heart of these mice was a result of the transgene expression, rather than uptake of secreted, recombinant enzyme. In contrast, the increased GAA activity in other organs such as liver, lung, and spleen (but not kidney and brain) reflected the uptake of the protein secreted from muscle source. Of note, only 20-25% of the wild type activity in liver was sufficient to completely clear glycogen. Thus, skeletal muscle can secrete GAA enzyme, and the enzyme can be taken up by distant organs—but the achievement of therapeutic enzyme levels, does not seem realistic. Importantly, there seem to be a risk of “overdose”; extremely high level of GAA expression in skeletal muscle (high expresser line) were not well tolerated, and virtually every cell contained small lysosomes with PAS positive material.

Interestingly, in vivo muscle mediated gene therapy of other lysosomal diseases was not very promising either. MV-mediated skeletal muscle gene transfer (human β-glucuronidase) in neonatal mice with the lysosomal storage disease mucopolysaccharidosis type VII, showed that the secretion of the enzyme from an intramuscular source was inefficient (Daly et al., 1999). Similarly, no enzymatic activity was found in serum or peripheral organs in hexosaminidase A-deficient knockout mice (Tay-Sachs disease) after skeletal muscle AV-gene therapy (Guidotti et al., 1999). It is possible, that lysosomal disorders, in general, are not good candidates for muscle mediated gene delivery.

In contrast, an efficient hexosaminidase secretion and restoration of enzyme activity was observed after liver transduction (Guidotti et al., 1999). Similarly, hepatic targeting of an AV-vector encoding GAA resulted in systemic correction in the 2 months old knockout mice (Amalfitano et al., 1999; Pauly et al., 2001). Our data show that the liver is an extremely effective organ for overexpression and secretion of the recombinant GAA protein into the bloodstream, which is taken up by peripheral tissues. Morphologically, the liver showed no abnormalities, indicating that local overexpression of GAA did not induce any pathological events. Comparable levels of transgene expression in both the muscle and liver result in a dramatically different amount of secreted protein—100 times greater in the plasma of the Alb-line. In the liver line, only the 110 kDa precursor, but not the processed forms, were detected in plasma, indicating that there were no liver cells damage. Furthermore, the enzyme was taken up by skeletal muscle and heart, targeted to the lysosomes, and prevented glycogen accumulation.

It is not clear why there is such a difference in the level of secreted GAA from skeletal muscle and liver. GAA is normally an intracellular enzyme, and the mechanism by which small portion of the synthesized enzyme is secreted is not well understood. Not to be limited by theory, it is possible, that a secretory pathway may not be efficient in skeletal muscle compared to liver cells. There are several implication from these studies: (1) liver cells could provide a source of the recombinant protein for enzyme replacement therapy; (2) both skeletal muscle and heart have significant capacity to up-take the protein as long as it is properly processed; and (3) a much lower level of hGAA expression in liver may be sufficient to provide correction in distant organs, since the levels of hGAA activity in skeletal muscle and heart were significantly higher than needed to remain phenotypically normal.

The following references have been cited in the specification and are hereby incorporated by reference.

Amalfitano et al., “Systemic correction of the muscle disorder glycogen storage disease type II after hepatic targeting of a modified adenovirus vector encoding human acid a-glucosidase,” Proc. Nati. Acad. Sci. USA, 96: 8861-8866 (1999).

Bijvoet et al., “Human acid α-glucosidase from rabbit milk has therapeutic effect in mice with glycogen storage disease type II,” Hum. Mol. Genet., 8: 2145-2153 (1999).

Blau et al., “Muscle-mediated gene therapy,” N. Engl. J. Med., 333: 1554-1556 (1995).

Chen et al., “Towards a molecular therapy for glycogen storage disease type II (Pompe disease),” Molecular Medicine Today, 6: 245-251 (2000).

Daly et al., “Neonatal intramuskular injection with recombinant adeno-associated virus results in prolonged β-lucuronidase expression in situ and correction of liver pathology in mucopolysaccharidosis type VII mice,” Human Gene Therapy, 10: 85-94 (1999).

Fuller et al., “Isolation and characterisation of a recombinant precursor form of lysosomal acid a-glucosidase,” Eur. J. Biochem., 234: 903-909 (1995).

Furth et al., “Temporal control of gene expression in transgenic mice by a tetracycline-responsive promoter,” Proc. NatI. Acad. Sci. USA, 91: 9302-9306 (1994).

Gros et al., “Insulin production by engineered muscle cells,” Human Gene Therapy, 10: 1207-1217 (1999).

Guidotti et al., “Adenoviral gene therapy of the Tay-Sachs disease in hexosaminidase A-deficient knock-out mice,” Human Molecular Genetics, 8: 831-838 (1999).

Hasse et al., “Gene therapy of murine neuron disease using adenoviral vectors for neurotrophic factors,” Nature Medicine, 3: 429-436 (1997).

Hermans et al., J. Biol. Chem., 266:13507-13512 (1991).

Herzog et al., “Stable gene transfer and expression of human blood coagulation factor IX after intramuscular injection of recombinant adeno-associated virus,” Proc. Natl. Acad. Sci. USA, 94: 5804-5809 (1997).

Hirschhorn, “Glycogen storage disease type II acid α-glucosidase (acid maltase) deficiency” in The Metabolic and Molecular Basis of Inherited Disease (Scriver, C. R., Beaudet, A. L., Sly W. S, Valle, D., Eds.) McGraw-Hill, pp 2443-2464, 1995.

Hoefsloot et al., “Expression and routing of human lysosomal alpha-glucosidasein transiently transfectedmammalian cells,” Biochem. J., 272: 485-492 (1990).

Kessler et al., “Gene delivery to skeletal muscle results in sustained expression and systemic delivery of a therapeutic protein,” Proc. Natl. Acad. Sci. USA, 93: 14082-14087 (1996).

Kikuchi et al., “Clinical and metabolic correction of Pompe disease by enzyme therapy in acid maltase deficient quail.” J. Clin. Invest., 101: 827-833 (1998).

Kistner et al., “Doxycycline-mediated quantitative and tissue-specific control of gene expression in transgenic mice,” Proc. NatI. Acad. Sci. USA, 93: 10933-10938 (1996).

Neufeld, “Lysosomal storage diseases,” Ann.Rev.Biochem., 60: 257-280 (1991).

Nicolino et al., “Adenovirus-mediated transfer of the acid alpha-glucosidase gene into fibroblasts, myoblasts and nyotubes from patients with glycogen storage disease type II leads to high level expression of enzyme and corrects glycogen accumulation,” Hum. Mol. Genet., 7: 1695-1702 (1998).

Partridge et al., “Myoblast-based gene therapies,” Br. Med. Bull., 51: 123-137 (1995).

Pauly et al., “Complete correction of acid alpha-glucosidase deficiency in Pompe disease fibroblasts in vitro, and lysosomally targeted expression in neonatal rat cardiac and skeletal muscle,” Gene Therapy, 5: 473-480 (1998).

Pauly, et al., “Intracellular transfer of the virally derived precursor form of acid alpha-glucosidase corrects the enzyme deficiency in inherited cardioskeletal myopathy Pompe disease,” Human Gene Therapy, 12: 527-538 (2001).

Poenaru, “Approach to gene therapy of glycogenosis type II (Pompe disease),” Molecular Genetics and Metabolism, 70: 163-169 (2000).

Raben et al., “Targeted disruption of the acid α-glucosidase gene in mice causes an illness with critical features of both infantile and adult humam glycogen storage disease type II,” J. Biol. Chem., 273: 19086-19092 (1998).

Raben et al., “Modulation of disease severity in mice with targeted disruption of the acid α-glucosidase gene,” Neuromuscular Disorders USA, 10: 283-291 (2000).

Recillas-Targa et al., “Positional enhancer-blocking activity of the chicken β-globin insulator in transiently transfected cells,” Proc. Natl. Acad. Sci. USA, 96:14354-14359 (1999).

Reuser et al., “Uptake and stability of human and bovine acid alpha-glucosidase in cultured fibroblasts and skeletal muscle cells from glycogenosis type II patients,” Exp. Cell Res., 155: 178-189 (1984).

Reuser et al., “Glycogenosis type II(acid maltase deficiency),” Muscle and Nerve Suppl., 3: S61-S69 (1995).

Tsuji et al., “Intracellular transport of acid alpha-glucosidase in human fibroblasts: evidence for involvement of phosphomannosyl receptor-independent system,” J. Biochem. (Tokyo), 104: 276-278 (1998).

Tsujino et al., “Adenovirus-mediated transfer of human acid maltase gene reduces glycogen accumulation in skeletal muscle of Japanese quail with acid maltase deficiency,” Human Gene Therapy, 9: 1609-1616 (1998).

Umapathysivam et al., “Determination of acid α-glucosidase protein: evaluation as a screning marker for Pompe disease and other lysosomal storage disorders,” Clinical Chemistry, 46:1318-1325 (2000).

Van den Hout et al., “Recombinant human α-glucosidase from rabbit milk in Pompe patients,” The Lancet, 356: 397-398 (2000).

Van der Ploeg et al., “Receptor-mediaterd uptake of acid alpha-glucosidase corrects lysosomal glycogen storage in cultured skeletal muscle,” Pediatr. Res., 24: 90-94 (1988).

Van der Ploeg et al., “Intravenous administration of phosphorylated acid alpha-glucosidase leads to uptake of enzyme in heart and skeletal muscle of mice,” J. Clin. Invest., 87: 513-518 (1991).

Van Hove et al., “High level production of recombinant human lysosomal acid α-glucosidase in Chinese hamster ovary cells which targets to heart muscle and corrects glycogen accumulation in fibroblasts from patients with pompe disease,” Proc. Natl. Acad. Sci. USA, 93: 65-70 (1966).

Vincent-Lacaze et al., “Structure of adeno-associated virus vector DNA following transduction of the skeletal muscle,” Journal of Virology, 73: 1949-1955 (1999).

Wisselaar et al., “Structurel and functional changes of lysosomal acid alpha-glucosidase during intracellular transport and maturation,” J. Biol. Chem., 268: 2223-2231 (1993).

Zaretsky et al., “Retroviral transfer of acid alpha-glucosidase cDNA to enzyme-deficient myoblasts results in phenotypic spread of the genotypic correction by both secretion and fusion,” Human Gene Therapy, 8: 1555-1563 (1997).

We claim:

1. A method for preparing a lysosomal enzyme suitable for replacement therapy, said method comprising:
(a) providing an expression vector effective for expressing the lysosomal enzyme in liver cells;
(b) introducing the expression vector into liver cells;
(c) expressing the lysosomal enzyme in the liver cells; and
(d) collecting the lysosomal enzyme.

2. The method of claim 1, wherein the lysosomal enzyme is acid alpha-glucosidase.

3. The method of claim 1, wherein the expressing step is performed in vitro.

4. The method of claim 3, wherein the introducing step is performed in vitro.

5. The method of claim 2, wherein the expressing step is performed in vitro.

6. The method of claim 5, wherein the introducing step is performed in vitro.

7. A lysosomal enzyme suitable for replacement therapy, said lysosomal enzyme being prepared by a method comprising:
(a) providing an expression vector effective for expressing the lysosomal enzyme in liver cells;
(b) introducing the expression vector into liver cells;
(c) expressing the lysosomal enzyme in liver cells; and
(d) collecting the lysosomal enzyme.

8. The lysosomal enzyme of claim 7, wherein the lysosomal enzyme is acid alpha-glucosidase.

9. The lysosomal enzyme of claim 7, wherein the expressing step and the introducing step are performed in vitro.

10. The lysosomal enzyme of claim 8, wherein the expressing step and the introducing step are performed in vitro.

11. A method for treating a deficiency of a lysosomal enzyme in a mammal, said method comprising:
(a) providing an expression vector effective for expressing the lysosomal enzyme in liver cells;
(b) introducing the expression vector into liver cells;
(c) expressing the lysosomal enzyme in liver cells; and
(d) introducing an effective amount of the lysosomal enzyme into the mammal.

12. The method of claim 11, wherein the lysosomal enzyme is acid alpha-glucosidase.

13. The method of claim 12, wherein the mammal is a human.

14. The method of claim 11, wherein the introducing step and the expressing step are performed in vitro.

15. The method of claim 12, wherein the introducing step and the expressing step are performed in vitro.

16. A method for treating a deficiency in a lysosomal enzyme in a mammal, said method comprising:
(a) providing an expression vector for the lysosomal enzyme which is effective for expressing the lysosomal enzyme in liver;
(b) introducing and presenting the expression vector to the liver of the mammal; and
(c) expressing the lysosomal enzyme in the liver, thereby treating the deficiency in the lysosomal enzyme.

17. The method of claim 16, wherein a regulatory factor is provided to regulate the expression of the lysosomal enzyme in the liver.

18. The method of claim 17, where the regulatory factor is provided orally.

19. The method of claim 18, wherein the regulatory factor is an antibiotic.

20. The method of claim 17, wherein the lysosomal enzyme is acid alpha-glucosidase.

21. A cultured mammalian liver cell transformed with an expression vector capable of expressing a lysosomal enzyme.

22. The cultured mammalian liver cell of claim 21, wherein the lysosomal enzyme is acid alpha-glutaminase.
